(19) United States
(12) Patent Application Publication
(10) Pub. No.: US 2026/0294771 A1
(43) Pub. Date: Oct. 1, 2026

(54) Topical Skincare Composition Containing Acadesine

(71) Applicant: Mark Thomas Mangano, Hillsborough, CA (US)

(72) Inventor: Mark Thomas Mangano, Hillsborough, CA (US)

(21) Appl. No.: 19/094,970

(22) Filed: Mar. 30, 2025

(51) Int. Cl. A61K 8/60; A61Q 19/00

(52) U.S. Cl. CPC A61K 8/606; A61Q 19/00

(57) ABSTRACT

A topical skincare formulation containing Acadesine, designed to activate AMP-activated protein kinase (AMPK) pathways in skin cells. The formulation improves hydration, enhances radiance, and supports cellular energy metabolism, representing a novel cosmetic application of Acadesine.

### BACKGROUND

The present invention relates to the use of Acadesine in topical skincare applications. Acadesine, an AMP-activated protein kinase (AMPK) activator, has been studied for systemic effects related to metabolism and cellular energy regulation. This invention explores its novel topical use in enhancing skin vitality, promoting microcirculation, and improving overall skin appearance.

### SUMMARY

Disclosed is a topical composition comprising Acadesine in a dermatologically acceptable carrier. The formulation is designed to improve skin radiance, hydration, and texture by activating cellular energy pathways when applied to the skin. This is the first known application of Acadesine in cosmetic skincare use.

### DETAILED DESCRIPTION

The topical composition includes Acadesine at a concentration ranging from 0.1% to 2.0% by weight. Preferred concentrations fall between 0.5% and 1.5%, depending on skin sensitivity and desired effect. Acadesine may be suspended in a carrier such as a cream, gel, serum, or emulsion designed to penetrate the epidermal barrier.

Upon topical application, Acadesine activates AMPK pathways in skin cells, leading to improved cellular metabolism, increased ATP production, and enhanced circulation. This results in visibly healthier, more radiant skin with reduced dullness and dryness.

1. A topical composition comprising Acadesine and a dermatologically acceptable carrier for application to human skin.

2. The composition of claim 1, wherein the composition is intended for non-therapeutic cosmetic enhancement of skin appearance.

3. A method of improving the appearance of skin comprising topically applying a composition containing Acadesine to a target area of the skin.
